(19) United States
(12) Patent Application Publication
(10) Pub. No.: US 2009/0041734 A1
(43) Pub. Date: Feb. 12, 2009

(54) USE OF CELL IMPLANTS IN BLADDER AND GUT OF NON-HUMAN ANIMALS FOR THE PRODUCTION OF PEPTIDES

(75) Inventors: Guido E.E.J.M. Van Den Thillart, Leiderdorp (NL); Herman P. Spaink, Oegstgeest (NL); Denhi Schnabel Peraza, Leiden (NL); Elmar S.D. De Pauw, Leiden (NL)

Correspondence Address: TRASK BRITT, P.O. BOX 2550, SALT LAKE CITY, UT, 84110, US

(73) Assignee: UNIVERSITEIT LEIDEN, LEIDEN (NL)

(21) Appl. No.: 11/885,136

(22) PCT Filed: Feb. 28, 2006
(86) PCT No.: PCT/NL2006/000104

§ 371 Date: Aug. 27, 2007

(30) Foreign Application Priority Data
Feb. 28, 2005 (EP) 05075490.2

(51) Int. Cl. A61K 35/00; C12N 5/06

(52) U.S. Cl. 424/93.21; 435/325

(57) ABSTRACT

Provided are means and method for the production of proteins in non-production in non-human animals. Cells producing a protein of interest is implanted in said non-human animal whereupon produced protein is harvested. In a preferred embodiment cells are implanted into a bladder or gut of said non-human animal

The invention relates to the field of peptide production by cell cultures. The invention in particular relates to the field of cell implants in unmodified cultured animals for production purposes.

Cell cultures are difficult or very expensive to use for the production of peptides due to many interfering factors such as infections, refreshing of media, control of end products, maintaining the quality of the producing cells and most importantly difficulty of creating a suitable environment for a cellular production system. The alternative, using genetically modified animals suffer from even more problems: cost of obtaining and keeping breeding quality, life cycle limits to get transgenic within a reasonable time frame, negative effects of the introduced transgenic on development and animal welfare, and most importantly the severe licensing restrictions to produce transgenic test animals, particularly vertebrates.

The present invention provides a method for the use of modified cells as cell implants in bladder and gut for the production of proteins allowing unimpaired excretion of proteins of interest. These products can be easily extracted from urine and faeces without handling the animals of different species. Species of animals can be chosen based the specific requirements of the produced proteins. In most instances, cost effective production can be achieved by using ectothermic species (i.e. fish, amphibians). The implantation and protein extraction can be automated.

The cells that are transplanted into the fish release one or more peptides. The cells may be transplanted in various ways as long as the secreted peptide(s) is (are) released into the excretory system and reach the external environment in sufficient quantity. The implants with peptide producing cells are preferably inserted into sites that have access to the excretion system. Preferred methods of insertion are injections of cells into the bladder and lower intestines at sites where protease activity is limited. Transplantation of cells is often used in mammals and a lot of experience has been obtained with respect to methods to transplant and maintain cells for at least some time. Important is that the cells remain present in sufficient numbers to allow sufficient and continuous production of the peptides. The cells may not be (or become) so numerous that they impede the development or the general health of the animal. In the mammalian world, several types of grafting aids have been developed to allow for prolonged stay of the cells or to allow differentiation of the cells. These aids can of course also be used in the present invention. Such aids include, but are not limited to, collagen or synthetic matrices for the grafting and attachment of cells. Also use can be made of co-injection of cells with enzymes, such as collagenases and other matrix metalloproteinases in order to improve settlement of the cells into the tissue of bladder and intestines. Alternatively, physical incisions of the surface of the tissue where cells are implanted can be used with a simple needle.

We would like to avoid long uncontrolled development of the cells and therefore in a preferred embodiment use predictable cell cultures for implantation. These cells could be obtained from the same species as primary cells that bare stabily transfected with a production vector or are tumour cells that produce a wanted peptide. As the cells, in this invention, need not be present for a very long time, it is possible to transplant fish cells from many different species into a recipient fish. If the evolutionary difference between the transplanted cells and the recipient is large, it is likely that the recipient fish will mount an immune response to the transplanted cells (the graft). However, as the cells often need only be present for a limited amount time, such immune response can typically be tolerated. To increase the robustness and predictability of the procedure it is preferred that the graft is derived from the same genus or family as the recipient organism. Preferably, the two are from the same species. As envisaged culture production organisms are typically outbred populations there are immunological differences between organisms of the same species. This is typically not a problem, however, it is possible to further match the graft and the recipient for common immunological markers. Typical markers are major and minor histocompatibility antigens. The grafting of the transplanted cells may further be facilitated by providing the recipient fish with immunosuppressants such as cyclosporin. Also in case use is made of vigorously growing tumour cell lines, various cytostatica such as compounds that are used for cancer chemotherapy can be administered at later stages of the production set-up to control the amount of implanted cells.

Preferred culture organisms are ectothermic species since they can be bred at wide range of temperatures and therefore need less maintenance costs. In addition, particularly for fish species many cheap production systems exist including specialized fish feeds and recirculation systems. In terms of space fish present the cheapest farming production system: most fishes have an ideal conversion factor of 1 (1 gram food leads to 1 gram of fish). Considering the many possibilities for species of cultured fish there can be many choices adapted to particular needs of production. For instance when bulk production of a compound is required use can be made of very large fish such as salmon or carp whereas, if a particular very highly specific compound has to be produced in a highly controlled manner in a short time, zebrafish can be used. For certain applications, when water volumes are limited, use can be made of amphibian culure animals such as bullfrogs or Xenopus. Normally the production lines works most cost-efficiently at room temperatures. For certain applications such as the implantation of human cell cultures it will be advantageous to use culture animals that can be bred at 37 centrigrades. In this respect many fish and amphibian species can be bred optimally at this temperature. In one embodiment of the invention use will be made of transparent culture animals such as embryos of fish or fish or amphibians that are transparent during adult live such as Chanda ranga or glass catfish. The advantage of a transparent production system is that cell growth of the implant can be monitored continuously by optical means from the outside. This invention is therefore also ideally suited to use for screening the potential of drugs for cancer growth inhibition. In this case the implanted cells are tumour cells which in a preferred embodiment of this invention are labelled with a tag that can be followed externally. Such tags comprise a fluorescent protein or an enzyme that can convert a compound into a fluorescent, luminescent or stained derivative. In this way tumour cell growth can be followed in high through-put by visual screening without any invasive technology. In this case, it is not necessary to implant the cells in bladder or gut.

In one embodiment of the present invention the food of the culture animal will be supplied by a resin that binds produced peptides released in the intestines in high capacity which further protects the peptides from degradation and simplifies the extraction of the product from the faeces.

In a preferred embodiment of the invention the culture animals are manipulated to a minimum extend, only once when injecting the cells. The waste products can be filtered and the water can be kept clean and sterile using UV light. When animals are fed by beads that bind the produced peptides the beads can easily be harvested by a simple filtering procedure.

Different aims of production are pharmacological products that in a preferred embodiment are peptides that are complexly glycosylated and therefore cannot be produced by non-vertebrates, i.e. plants, yeast and bacteria. For instance we can produce hormones by modified cells that are transplanted in our culture organisms. These hormones include human proteins as well as peptides from domesticed animals. Examples are Growth hormone, Corticoliberin (Adreno Corticotrope hormone), Thyroid stimulating hormone, Follicle-stimulating hormone (FSH), Luteinizing hormone (LH), Prolactin, Chorion gonadotropin(CG), MG (Menopause gonadotropin), Somatotropin, Prolactin, Placental lactogen, Calcitonin, Parathyroid hormone, Colony-Stimulating Factor (CSF), Epidermal growth factor (EGF), and Erythropoitine, or a combination thereof. The hormones mentioned above are also known under different names. As the underlying amino acid sequence is the same, the hormones referred to by the synonyms are also within the scope of the invention. For instance, Growth hormone is sometimes also referred to as Somatrotopin, somatotrophic hormone, hypophysis growth hormone, somatotropic hormone or STH. Corticoliberin is also referred to as releasing corticotropin hormone. Adreno Corticotrope hormone is also referred to as Corticotropin, adrenocorticotropin, adrenotropin, corticotropin, ACTH or adrenocorticotrophic hormone. Thyroid stimulating hormone is also referred to as TSH, thyrotropin or thyrotropic hormone. FSH is also referred to as Follicule stimulating hormone, follitropin or gametocinetic hormone. LH is also referred to as Luteinizing hormone, Luteotropin or interstitial cell stimulating hormone (ICSH). Prolactin is also referred to as PRL, lactogenic hormone, mammotrophic hormone, galactopoietic hormone or lactotrophin. CG (Chorion gonadotropin) is also referred to as Chorionic gonadotropic hormone, chorionic gonadotrophic hormone, choriogonadotropin or chorionic gonadotropin and Menopause gonadotropin (MG) is also referred to as urogonadotropin, menotropin or Menopause gonadotrophic hormone.

In addition many other peptides that are difficult or too expensive to produce in other ways can be efficiently produced in our invention. Such compounds include a wide scale of monoclonal antibodies, expensive enzymes that needed for therapies of patients, inhibitory peptides such as protease inhibitors.

It is possible that the recipient develops an immune response against a heterologous peptide. Although this immune response is typically too late to affect the production of the peptide, it can be advantageous to harvest the antibodies against the protein from the blood since these could have a value by themselves. The immune response of the cultured ectothermal animals is usually temperature dependent making this a very efficient way to control immune responses. For instance in cyprinid fish the immune system is inactive below 18 degrees Celsius.

The cells can either express the desired peptide without manipulation or can be manipulated to express the desired peptide. For instance human bladder tumour lines have been described that produce hCG (Cancer Res. 1995 Apr. 1; 55(7):1479-84. Nishimura R, et al). Since these lines are derived from bladder these are ideally suited for implantation into the bladder according to our invention. When the cells do not express the peptide already or do not express sufficient peptide, they can be provided with the genetic information for expressing the peptide.

In a preferred embodiment the cells are provided with the genetic information to express the peptide. This can be done by providing the cells with expression cassettes comprising coding sequences for the peptides. However, it is also possible to activate the endogenous genes by inserting an active regulatory sequence near the coding sequence(s) for the respective hormones. This can be done for instance through homologous recombination.

As an example how our invention is enabled in practise we describe the production of LH heterodimes in a cell implant system in fish as was carried out recently in our laboratory. The LH proteins belong to a family of related proteins that share the characteristic that they are functional as heterodimers consisting of a common α-subunit and a different β-subunit. In the case of hormones that consist of more than one protein chain it is possible that cells do not express all of the chains needed to generate the hormone. In these cases only expression cassettes are required for the chain(s) that are lacking. Thus, if one or more but not all of the subunits of the hormone are adequately expressed in the cells one only needs to express the remaining subunit(s) in the cell. If none of the subunits are expressed, one has to manipulate the cells such that all of the subunits are expressed at adequate levels. In a preferred embodiment, the cells are provided with expression cassettes for the subunits of the hormone. The cells are provided with expression cassettes for the two protein chains that make up LH (i.e. for the α-subunit and the unique β-subunits for the hormone), or in separate cell lines the combination of βLH+α are expressed. Thus in our enabled embodiment said cells are genetically modified to express said hormone(s). In a preferred embodiment, the cells are provided with one or more genes encoding peptides.

The cells can be primary cells or cell lines that are cultured in vitro for an extended period. The cells can also be tissue fertilized eggs or embryonic tissue. In a preferred embodiment, the cells are derived from a clonal population of cells. In this way, the cells can be subjected to detailed quality control prior to use. This also allows for the generation of cell banks that have the same property. Moreover, a clonal population can be subjected to further manipulations. For instance, if one wants to reduce immune responses in the recipient, it is possible to knock out expression of major and/or minor histocompatibility antigens. Thus in a preferred embodiment, the cells have been selected for a reduced immunogenicity in the recipient.

A method of the invention may be used for all types of culture animals. Preferred culture animals are fish that are typically used in aqua farming systems such as carp, salmon, goldfish, zebrafish, trout, catfish, sturgeons, seabream, seabass, tilapia, medaka, nile-perch, glass catfish and commonly cultured aquaria fish. However, the invention is suitable for all vertebrate culture animals.

The invention further provides an isolated and/or recombinant fish cell that produces a peptide. In a preferred embodiment said cell is genetically modified to express said peptide. The invention further provides a fish cell provided with the capacity to express a peptide that is optimally suited for transplantation purposes. Preferably, the fish cell is provided with a recombinant and/or isolated nucleic acid sequence encoding said peptide. If the peptide consists of one or more subunits, the fish cell is preferably provided with an isolated and/or recombinant nucleic acid sequence encoding at least one subunit of said peptide. Preferably, the fish cell is provided with nucleic acid sequence encoding all subunits of said peptide. In one embodiment of the present invention the cells are derived from the bladder or intestines of the culture animal species that is used for production. This gives the best warranty that these cells are inserted efficiently in the tissue without any immune responses that negatively influence the production of the implanted cells. Such cell cultures can be immortalized in order to provide vigorous growth capacity. using standard technology.

The invention further provides a culture animal that comprises a cell according to the invention. Preferably, said culture animal is a fish or amphibian for which standard aqua-cultural procedures exist. Preferably a tilapia and/or a catfish, preferably a Clarias gariepines.

The invention further provides a method for transplanting cells into a fish comprising anaesthetizing said fish, opening the papilla that connects the bladder and/or intestines of said fish with the surrounding water and injecting cells via the urethra and/or intestine into the intestinal and/or bladder lumen. Opening of the papilla can be performed in various ways. In a preferred embodiment of the invention, the papilla is cut to remove the tip of papilla. In a preferred embodiment, the papilla is cut such that at least a portion of the external part of the papilla is left. The number of cells to be inserted is not very crucial. Typically, however, at least 10⁵ cells are inserted. Preferably, at least 10⁶ cells are inserted. As mentioned previously, the cells infected into the fish or amphibian can be any animal cells. It can also be a mammalian cell, see for instance, Dev. Dynamics, 2333: 1560-1570 (2005). Preferably said method is used for attaching cells to the bladder.

The present invention further provides a fish or an amphibian transplanted with an animal cell. In one embodiment said fish is provided with a mammalian cell or with a fish cell. Further provided is a fish comprising an papilla connecting the bladder and/or intestines of said fish with the surrounding water, wherein the tip of said papilla has been removed and wherein said bladder is canulated through said papilla by a canule. Further provided is a method for obtaining a protein produced by cells transplanted in the bladder of a fish, comprising removing the tip of the papilla that connects the bladder and/or intestines of said fish with the surrounding water, canulating said bladder through said papilla using a canula, and harvesting urine from said fish via said canula.

In a preferred embodiment use is made of implants of cells that can be cultured outside the body of the animal. Furthermore these cells are preferably clonal, preferably selectable for characteristics. It is however also possible to make use of primary cultures, tissue, fertilized eggs, or embryonic material. A method for making transgenic fish eggs has been published (Morita et al 2004, Transgenic Research 13, 551). However, there are several advantages of using culturable cells above using fish eggs. 1) Transgenic fish eggs cannot be further propagated. 2) The expression of the introduced gene(s), as described by Morita et al, is not stable. 3) Selection of the introduced genes in eggs is difficult. 4) Using fish eggs as bioreactors is time consuming in relation to culturable cells because micro-injection for each of the eggs has to be used, whereas in cell-cultures we can make use of standard transfection technology. 5) Culturable cells can easily be stored, and made available at any time. 6) There are no ethical problems with working with culturable cells as compared to genetically modified eggs. 7) Injection of a suspension of cultured cells is rather easy as compared to implantation of eggs, and/or embryos, or tissues.

The results show that technology for implantation of cells of the present invention is successful and can be performed routinely. Cells efficiently attach themselves to the bladder of the host fish. Furthermore, the present invention demonstrates that fish urine is not detrimental to survival and growth of the attached cells. The cells survive the presumably rather harsh environment of the fish urine and are able to grown and remain metabolically active as shown by the production of β-galactosidase.

The invention further provides a method for collecting urine from a fish comprising implanting cells in said fish, preferably in the bladder and after cell implantation and allowing for some time of growth of implanted cells, preferably at least 12 hours, ore preferably at least one day, preferably at least 4 days. In one embodiment fish are incubated in water with a composition that stimulates a fase of low urine production in the fish. Preferably said fish are maintained in water of 300 mosm (for example containing 1% sodium chloride) preferably for at least 6 hours, more preferably at least 24 hours, preferably at least 4 days. Optimal timing depends on the fish species, for tilapia at least 3 days is possible without harming the health of the fish. The incubation will result in very low urine production. After transferring the fish to fresh water (i.e. without added salt) a high urine production follows that can subsequently be collected by a canule (this can be attached permanently to the fish as mentioned in the legend of FIG. 14). In this way a controlled product release is achieved. Since urine has a very low content of contaminating proteins and represents a normally sterile environment, there is little effort involved in purifying a peptide product produced by the implanted cells. For instance, a simple dialysis procedure allows to get rid of the contaminating (low molecular weight) urine waste products. A simple concentration using a centrifuge tube containing a molecular weight filter (e.g. Centricon technology) already delivers a rather pure product. Therefore this method can be considered cost effective compared to purification of products produced by cell cultures. These cells cultures contain growth factors that are hard to get rid of. Another advantage of our method is that we don't need to add artificial growth factors. Such growth factor, such as fetal calf serum can be contaminated with diseases such as prion disease that are highly unwanted for any application and very difficult to get rid of. In contrast fish products are generally considered save for consumption of medical applications (e.g. there are no prion related disease known in fish that can be transferred to other organisms). Therefore for any consumption of medical applications our method can be of great advantage as well in costs as in quality over in-vitro cell cultures.

We also tested human cell implantations. This makes it possible to produce peptides that are post-translationally modified in a human fashion and therefore optimally suited for human biomedical applications. Although we only tested a human primary pancreas tumor cell line, it could be more advantageous to use human bladder tumor cells that are better adapted to the bladder environment. Thee present provides a fish or an amphibian comprising a mammalian cell, preferably a human cell. Melanoma cells, for instance, proliferate in zebrafish Lee et al, (Dev. Dynamics, 2333:1560-1570, (2005). The present invention therefore further provides the use of fish and/or amphibians as a screening system for anticancer drugs. In a preferred embodiment said cells are implanted in the bladder of said fish or said amphibian. The presence of the tumor cells can be detected in the fish by a simple analysis of the urine, for instance by means of mass spectrometry and for instance the detection of human peptides. Since human peptides can easily be discriminated from fish peptides by standard mass spectrometric analysis. The invention therefore provides implantation of human tumor cells in the bladder of a fish and subsequent, preferably periodic, urine collection. This can be used to determine whether the tumor cells are still present. This is useful, for instance, to determine whether the tumor responds to a “potential” anti-tumor therapy such as a drug. Such a drug is preferably added to the water of the fish (many compounds are taken up by fish through the gills or mouth). For instance, steroids are easily taken up by gills. Some of the more potent anti-tumor drugs are steroids. The urine tests show whether the drug are effective. Alternatively, if the chemical compound is expected to be poorly transmissible through tissue, then the compound is provided directly into the bladder via the or a canule (as described in FIG. 14). Samples of the urine after treatment with the antitumor drug will be analyzed for the presence of human peptides providing a simple read-out method for the activity of the drug on the tumor cells. An advantage of a method of the invention over, for instance, screening of antitumor compounds in cell cultures, is that in the present invention the cells are in a natural tissue environment in the absence of artificial growth factors (such as fetal calf serum) and therefore the effect of the antitumor drug is more predictive for the effect on tumors in patients. In a preferred embodiment a cell of the invention produces a marker that can be detected visually, for instance, a fluorescent protein. This embodiment is particularly suited for following the fate of implanted cells in transparent organisms such as preferably, fish embryos, transparent fish and frogs or any other transparent animal. This feature can for instance be used to follow tumor cells in transparent fish.

### EXAMPLES

### Example 1

### Cloning of LHβ, FSHβ and α zebrafish genes

The genes of zebrafish are already published LHβ (AY424304), FSHβ (AY424303) and α (AY424306 , also the genes of eel are reported for LHβ (AB175835) in Anguilla japonica; FSHβ (AY169722) in Anguilla anguilla and for α (AB175834) in Anguilla japonica. In order to clone LHβ, FSHβ and α primers were designed based on the cDNA sequence of each one:

The sequence used for the design of the primers of the LHβ was:

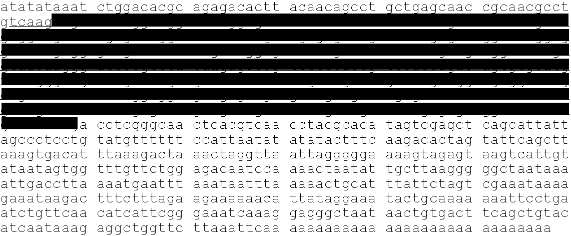

The sequence used for the design of the primers of the FSHβ was:

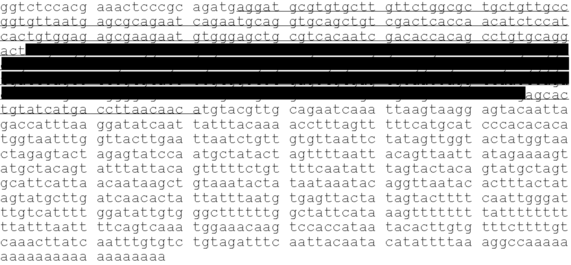

The sequence used for the design of the primers of the α subunit
was:

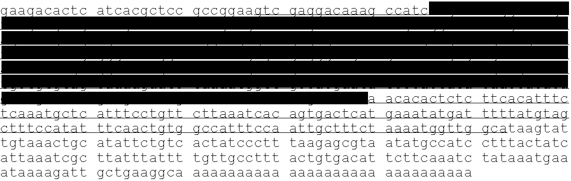

In each case the blue part represents the coding sequence, and the predicted amplified region is underlined.

The primers designed were:

Upper-LHβ/EcoRI
5′-CAA CCG AAT TCA ACG CCT TCA AGA TGT-3′
Lower-LHβ/EcoRV
5′-CCG ATA TCT AGT ATG CGG GGA AAT-3′
Upper-FSHβ
5′-AGG ATG CGT GTG CTT GTT CT-3′
Lower-FSHβ2/3
5′-TGT TGT TAA GGT CAT GAT ACA GTG C-3′
Upper-α1
5′-GTC GAG GAC AAA GCC ATC AT-3′
Lower-α1
5′-TGC CAA CCA TTT TAG AAA CGA-3′

To facilitate further cloning steps the LHβ oligos, include restriction enzyme sites for EcoRI and EcoRV in the upper oligo and in the lower oligo respectively.

Total RNA was isolated from zebrafish heads homogenized in liquid nitrogen and extracted using TRIZOL reagent according to the manufacturer's instructions. Traces of DNA were removed by incubation with DNaseI followed by phenol/chloroform extraction and ethanol precipitation. RT-PCR was performed using the Superscript II one step RT-PCR system with platinum Taq. Reactions were performed with 100 ng of total RNA using 25 pmol of the upper and lower primers. Reverse transcription was performed at 50° C. for 30 min. PCR conditions were 40 cycles of denaturation at 94° C. for 20 s, annealing at and 55° C. for LHβ and for FSHβ and 50° C. for a during 30 s and extension at 72° C. for 1 min followed by a final extension step at 72° C. for 10 min. The PCR products were separated by electrophoresis in a 1% gel of agarose and stained with ethidium bromide. The FSHβ PCR product produced a faint band when observed in the agarose gel, in order to optimize we did a PCR using as template this PCR product the reaction was performed with 1/20 of the PCR product reaction using 10 μM of the upper an lower primers. PCR conditions were 40 cycles of denaturation at 94° C. for 20 s, annealing was performed in a gradient at from 50° C. to 60° C. for 30 s and extension at 72° C. for 1 min followed by a final extension step at 72° C. for 10 min. A sharp band was then observed. To confirm the identity of the amplified sequences, the PCR products were cloned in pCRII-TOPO vector, digested with restriction enzymes to identify the correct direction and sequenced. The analysis of the sequence revealed that we had cloned LHβ,α and FSHβ subunits of zebrafish. These constructs allow now sub-cloning the genes under a constitutive promoter.

Cloning LHβ, FSHβ and α under the control of a constitutive promoter (CMV).

The p3XFLAG-CMV-9 expression vector is used to establish transient or stable fusion proteins. The vector encodes three adjacent FLAG epitopes upstream from the multicloning region. This results in an increased detection using anti-FLAG antibody. The promoter-regulatory region of the CMV drives transcription of flag fusion constructs. The preprotrypsin leader sequence precedes the FLAG sequence, promoting the secretion of the protein. The amino glycoside phosphotransferase gene (Neo) confers resistance to amino glycosides such as Geneticin (G418), allowing for selection of stable transfectants.

We used these vector because it has the advantages that the synthesized proteins will be secreted driven by the preprotrypsin leader, we can detect the expression of the proteins by western blots with the anti-FLAG antibody and we can make stable cell lines selecting with geneticin.

Cloning of LHβ

The PCR product was purified and digested with EcoRI and EcoRV as well as the p3XFLAG-CMV-9 expression vector. The DNA fragments were ligated overnight at 4° C. The ligation mixture was then used to transform chemical competent cells. Enzymatic digestions selected positive clones. Those clones that give a correct pattern of digestion were sequenced. At the moment, the clone sequenced showed an incorrect insertion of LHβ so I had to repeat this cloning step. The predicted aminoacid sequence gives a protein of 187 amino acids with a molecular weight of 20593.8

Cloning of FSHβ

pCRII-TOPO FSHβ was digested with BamHI/NotI the band that corresponds to FSHβ cDNA was purified and sub-cloned in the p3XFLAG-CMV-9. The positive clones were then digested then with Not/EcoRV and the mug bean nuclease and religated to get FSHβ in frame with the preprotrypsin leader, these construct was designated as CMV-FSHβ. Alternatively, pCRII-TOPO FSHβ was digested with Xba/BamHI and the resulting band was subcloned in the p3XFLAG-CMV-9. The positive clones were then digested then with EcoRV and religated to get FSHβ in frame with the preprotrypsin leader, this construct was designated as CMV-FSHβ. Enzymatic digestions selected positive clones; those clones that give a correct pattern of digestion were sequenced. The sequencing of the two different colonies, which correspond to the different strategies of cloning, was correct. These constructs were used for the transformation of the ZF4 cell line. The predicted amino acid sequence gives a protein of 174/183 amino acids with a molecular weight of 19062.24/19965.21 respectively to the two different strategies of cloning.

Cloning of α

pCRII-TOPO α was digested with KpnI/XbaI and the resulting band was subcloned in the p3XFLAG-CMV-9. Enzymatic digestions selected positive clones. Those clones that give a correct pattern of digestion were sequenced. The analysis of the sequence revealed that we have cloned a in the correct orientation and in frame with the flag and the preprotrypsin leader sequence this construct received the name CMV-α. The predicted amino acid sequence gives a protein of 190 amino acids with a molecular weight of 21083.24

The plasmids CMV-FSHβ CMV-FSHβ and CMV-α were purified by alkaline lysis with SDS using the QIAprep spin Miniprep kit. The purified plasmids were linearized with ScaI. The linearized products were separated by electrophoresis in a 1% gel of agarose and stained with ethidium bromide. The linearized plasmid was purified and quantified.

Transfection of FSHβ in zebrafish fibroblast cell line (ZF4) The ZF4 (ATCC number: CRL-2050) cells are fibroblast from 1 day-old zebrafish embryos. The frozen aliquot of ZF4 cells was removed from the liquid nitrogen and placed immediately on ice for 10 minutes. The thawed cell suspension is removed from the vial and diluted in 10 ml of complete medium (1:1 mixture of Dubelco's modified Eagle's medium and Ham's F12 containing 1.2 g/L of sodium bicarbonate, 2.5 mM L-glutamine, 15 mM HEPES and 0.5 mM sodium pyruvate, 10% of fetal bovine serum and penicillin/streptomycin) at room temperature. The supernatant is discarded by centrifugation at 1200 rpm for 8 min. The cells are resuspended in 8 ml of complete medium and transferred to a tissue flask (T25) and cultured at 28° C. The cells are examined under the inverted microscope to check for cell density. In cultures with a confluence of 80% the medium is removed and washed with 3 ml of PBS to remove cellular debris and serum. Then 0.5 ml of trypsin solution (0.25%) is added and incubated at room temperature until the cells from the monolayer detach from the flask, when a single cell suspension has been obtained 5 ml of complete medium is added to stop trypsinisation. Viable and nonviable cells are counted using a Fuchs-Rosenthal hemocytometer. Cells were seeded in new flasks at a density of 100-150 cells/mm² (5×10⁵ in each T25 flask). The cells were incubated at 28° C. for maximum 4 days before the next passage. Transfection was realized in zebrafish fibroblasts when they were 50-60% confluent using Fugene 6 following manufacturer's instructions. The Fugene6/DNA complex is prepared in a 6:1 ratio of Fugene6: DNA in medium without serum. The culture medium is removed from the cells and replaced with serum free medium. The Fugene6/DNA complex is added drop wisely and mixed. The cells are incubated at 28° C. for 5 hours. Then the medium is removed and replaced with complete medium. As a control Zf4 cell were co-transfected with the pEYFP-N1 plasmid and analyzed for positive cells under the Leica confocal at 16 and 24 hours after transfection. Different concentrations of DNA were used to establish the optimal concentration for this plasmid giving as a result that 1 μg of DNA in a surface of 21 cm² was the best concentration to obtain a transformation of approximately 30%.

Detection of the Expression of LHβ, FSHβ and α

Immunohistochemistry

Transfected cells were analyzed also by immunohistochemistry to detect the expression of the protein in the cells. The cells were fixated with p-formaldehyde 2% glutaraldehyde 0.1% in PBS during 10 minutes, to eliminate the fixative cells are washed twice with PBS. Then they were permeabilized for 10 minutes with 0.2% of Triton X-100 in PBS. To reduce auto-fluorescence caused by the fixative NaBH4 2 mg/ml in PBS was added during 10 min. Blocking was done with 0.1%BSA-c, 0.02% cold water fish skin gelatin during 30 minutes. Incubation with the anti-FLAG antibody (1:250) was performed overnight at 4° C. in 0.1%BSA-c, 0.02% cold water fish skin gelatin. The antibody was removed and washed with 0.1%BSA-c, 0.02% cold water fish skin gelatin twice 10 min each. The secondary antibody (anti-Rabbit Alexa 488) is added in a dilution 1:1000 in 0.1%BSA-c, 0.02% cold-water fish skin gelatin and incubated at room temperature during 60 min. Then is washed three times with 0.1%BSA-c, 0.02% cold water fish skin gelatin and mount in DABCO/Gelvatol. The analysis of the cells in the Leica confocal revealed expression of the protein in vesicles in the transfected cells. This localization is in agreement with the expected site for proteins that are going to be secreted.

Western Blot

The different constructs were transfected using Fugene 6 in T25 flasks in duplicate, as control cells were not transfected with DNA, transfected with the empty vector and with the positive control CMV-BAP. The proteins were obtained at the third and fifth day after transfection from the supernatant. The supernatant was concentrated using the amicon columns following the manufacturers instructions. FLAG fusion proteins were immunoprecipitated with Anti-FLAG M2 affinity gel, following manufacturers instructions. Proteins samples were diluted 1:4 with sample buffer boiled and kept at −80° C. For setting the conditions of the western blot only the positive (CMV-BAP) and negative (NO DNA) controls were analyzed, samples were loaded in a 12% acrilamide gel, run during 60 min at 50 mAmp. The gels were blotted in nitrocellulose membranes. Membrane was blocked with milk 5% overnight at 4° C. and immunodetection was realized against anti FLAG antibody at a 1:250 dilution in milk 5% during one hour at room temperature. Immunodetection was revealed with ECL following manufacturer's instructions. The antibody recognized the expressed BAP protein showing a band of the predicted size. With these experiments we show that transient transfected cells are producing the proteins of interest.

### Making Stable Cell Lines

Cells were transfected in 6 well chambers with linearized and purified DNA using Fugene 6. As a control cells were transfected without DNA. After 3 or 5 day after transfection the complete medium was replaced with complete medium with G418 added. The amount of G418 to kill cells that are not expressing the construct varies from cell line to cell line. For the ZF4 cell line the concentrations suggested by the manufacturer are between 0.8 and 1 mg/ml. Cells were treated with 0.8 and 1 mg/ml in quadruplicates, media was changed daily to wash out the dead cells. This was done during 15 days until the plates that contained the cells that did not have DNA were dead and we could not observe any cell in the plate. Then the transfected cells were incubated with the same concentration of G418 for another 5 days. When a confluent monolayer was obtained, the cells were subcultured into a T25 flask and the concentration of G418 was lowered to 0.5 mg/ml in complete media. Stable cell lines will be tested with immunohistochemistry and western blot analysis with anti-FLAG antibody to detect the expression of the protein.

Bioassay

In order to test if the hormones expressed by the transfected cells are active a simple bioassay is going to be performed. Follicular cells cultures from zebrafish respond to pituitary extracts and/or human corionic gonadotropin (hCG) by upper or down regulating the expression of different genes. hCG (15 IU/ml) increases the expression level of activin βA in a time dependent manner. This effect is evident at 40 min of the treatment and reached a maximal level at 2 h, longer treatment (4 h) causes a diminishment of the effect. In contrast activin βB is suppressed in the same conditions. When experiments using different concentrations of hCG are performed a dose dependent respond is observed. Goldfish pituitary extract also stimulates expression of activin βA and suppresses activin βB in a dose dependent manner. We plan to use this characteristic of the follicular cells in culture to test if the FSHβ and LHfβ are active.

In Vitro Follicular Cell Culture

Isolation of Follicular Cells

Young zebrafish were purchased from a pet store and maintained without separation of males and females. Females were anaesthetized with 0.01% tricaine methansulfonate solution for 2 minutes or until they were standing still, and decapitated before dissection. The ovaries were then removed and placed in a 10 mm culture dish with L-15 (Gibco). The follicles from 5 females were carefully separated with the aid of insulin needles. The separated follicles were measured with an ocular micrometer in a dissecting microscope and the healthy viotellogenic follicles around 0.45 mm were selected, pooled and cultured in T25 flask for 6 days in M199 medium supplemented with 10% fetal bovine serum at 28° C. and 5% CO₂. The medium is changed on the third day of the incubation. During the 6-day incubation follicle cells proliferated significantly, increasing the yield of cells for the experiments. Cells are washed and trypsinised at 28° C. for 15 min. Thereafter the cells are washed three times with medium M199 through centrifugation at 1000 rpm for 2 min, and then subcultured in 24 plate at a density of 1×105 cells/ml per well for 24 hours in complete M199 before hormone treatment. The amount of cells was not enough for the experiment, so we should start with 20 females to get enough material.

Hormone Treatment

Different concentrations of the supernatant will be used, as a positive control hCG will be used at a 15 IU/ml, and carp pituitary extracts will also be included. With the pituitary extract condition we can compare the amount of cells that should be used to observe an effect in the zebrafish bioassay.

In vivo Injection

hCG was dissolved in 0.9% of NaCl solution in a concentration of 20 IU/ml. Each fish will receive 50 μl of saline as a negative control, hCG as a positive control and different concentrations of the supernatant or the purified FSHβ and/or LHβ. At 1, 2, 4, 6 and 12 h after injections fish are killed and ovaries removed for RNA extraction

RNA Extraction

At the end of the hormonal treatment total RNA was isolated from zebrafish ovaries or follicular cells, homogenized in liquid nitrogen and extracted using TRIZOL reagent according to the manufacturer's instructions. Traces of DNA were removed by incubation with DNaseI followed by phenol/chloroform extraction and ethanol precipitation. RT-PCR was performed using the Superscript II one step RT-PCR system with platinum Taq. Reactions were performed with 100 ng of total RNA using 25 pmol of the upper and lower primers. Reverse transcription was performed at 50° C. for 30 min. PCR conditions were 40 cycles of denaturation at 94° C. for 20 s, annealing at 56° C. during 30 s and extension at 72° C. for 1 min followed by a final extension step at 72° C. for 10 min. The PCR products were separated by electrophoresis in a 1% gel of agarose and stained with ethidium bromide.

Designed primers to amplify Activin βA, Activin βB and βActin:

| | |
|---|---|
| Upper-Activin A | 5′-TGC TGC AAG CGA CAA TTT TA-3′ |
| Lower-Activin A | 5′-CAT TCG TTT CGG ACT CAA G-3′ |
| Upper-Activin B | 5′-CAA CTT AGA TGG ACA CGC TG-3′ |
| Lower-Activin B | 5′-GTG GAT GTC GAG GTC TTG TC-3′ |
| Upper-βActin | 5′-CCC CTT GTT CAC AAT AAC CT-3′ |
| Lower-βActin | 5′-TCT GTG GCT TTG GGA TTC A-3′ |

Transplant of the stable cell lines in the fish:

Stable cell lines will be transplanted to fish bladders. Fish will be anesthetized and an injection will be given of cells into the bladder or intestines. The correct amount of cells that produce a constant amount of hormone will be transplanted. As a negative control cells that do not express hormones and only have the empty vector will be injected and as positive control a group of fish will be injected in the bladder or intestines with purified LH. A third testing condition regards the extraction of the excreted protein from the urine and injection of this product in fish for controlling the response on the oocyte. This will be done with the bioassay that we developed as described above.

### Example 2

Implants with hormone producing cells: Maturation response of silver eels

Zebrafish ZF4-cells transfected with β-galactosidase, LH and FSH genes were brought into stable cell lines. Silver eels in the migratory phase were injected with a mixture of these hormone producing cells (HPC) or stimulated with weekly injections of carp pituitary extracts (CPE). Over 4 weeks eels were sampled and maturation parameters were analyzed. A correspondence between the two stimulation methods was observed for eye index, and pectoral fin index. Thus ZF4 cells were not rejected but even continue to grow in the transplanted animals. The transplanted genetically modified cells have the capacity to produce and secrete hormones in the fish for an extended period of time. The transplanted cells promote sexual maturation of the transplanted fish.

Reproduction is severely depressed in silver eels. A Japanese protocol for artificial reproduction takes 3-6 months when applied on European eels (Palstra et al 2005). This extremely long induction period for maturation is related to the fact that silver eels are still in their pre-pubertal stage and that the secondary vitellogenesis is not at all developed in silver eels. This process is apparently even more depressed in European eels. An approach to circumvent the time consuming and stressful weekly injections, is to implant hormone producing cells in silver eels, and leave the animals without further disturbance.

In this experiment we compared the effect of the weekly injections with CPE (carp pituitary extract) with a single injection with hormone producing cells. Hormone producing cells used in a method of the invention allow lower, constant hormone levels, and lower stress levels (no injections), which improves the quality of the mature eels. Zebrafish cell lines injected in eels provide evidence that maturation is initiated that way and is compared with stimulation by CPE injections.

### MATERIALS AND METHODS

Silver Eels

Wild migratory silver eels were caught in Lake Grevelingen near the sluices at the North Sea side (at 32 ppt, 12° C.) at the end of October 2005. Larger eels (>70 cm and 800 g) were selected from the catch. They were transported by car to the laboratory in a 50:50% eel:water ratio in plastic bags filled up with oxygen in a 60 l tank. In total 73 randomly chosen silver eels were used for data collection; 10 eels were immediately measured and sampled as controls, another 10 eels were measured and sampled as rest-group at the end of the experiment. For hormonal stimulation 53 experimental eels were tagged with small passive transponders (TROVAN, EID Aalten BV, Aalten, The Netherlands).

Treatment

A group of 27 eels received weekly Carp Pituitary Extract (CPE) injections at a dose of 20 mg/kg according to the method described before (Palstra et al., 2005), referred to as the CPE-group. In total, 26 eels received at the start of the experiment 1 ml of a mix of 4 types of cells (β-gal, LH β, FSH β, LH/FSHα), referred to as the Hormone Producing Cells (HPC) group. The cells were injected as a suspension subcutaneously below the beginning of the dorsal fin and above the lateral line. The CPE and HPC groups were kept in separate 1,700-l recirculation systems with artificial seawater of 18° C. To prevent bacterial infections, eels from both groups were exposed weekly for 1.5 hours to the antibiotic Flumequin (Flumix, Eurovet, Bladel, The Netherlands, both of 50 mgl⁻¹ for 1-2 h) in 200-l water in a separate tank.

Measurements & Sampling

Each week, 5 or 6 eels from each group were measured and sampled to analyse the treatment effects. Bodylength (BL in cm), gonad (G), liver (L), and body weight (BW in g) were measured and used to determine:
Fulton's condition factor K=100*BW/BL³
Eye index (Pankhurst, 1982) EI=100*((EDh+EDv)/4)²π/10*BL)
Pectoral fin length index (Durif et al. 2005) PFLI=100*PFL/BL
Silver index based on BL, BW, ED and PFL, see Durif et al. (2005).
Gonadosomatic index (GSI): (Weight gonads/Body weight)*100%

Blood (500 μl) was taken from the caudal vein with heparin flushed (10.000 IU/ml) 1-ml syringes kept on ice. Haematocrit (Hct) was measured in 9 μl whole blood samples in triplicate using a micro-centrifuge (Bayer, F.R.G.). Haemoglobin (Hb) was determined in 10 μl in duplicates with a LS50B, Perkin Elmer spectrophotometer at a fixed λ of 550 nm using the MPR 3 kit (1 ml, Roche Diagnostics GmbH). The MCHC (Mean Cellular Haemoglobin Content) was calculated from ratio Hb/Hct. Blood was then centrifuged for 5 min at 14,000 rpm and blood plasma was stored at −80° C.

β-galactosidase Tissue Staining

Tissue from the place of injection including skin, fat tissue and muscle tissue was obtained after 1, 2, 3 and 4 weeks after the injection. The tissue was rinsed in PBS briefly and fixed immediately with paraformaldehyde 1%-glutaraldeyde 0.1% in PBS with MgCl2 2 mM, EDTA 5mM and NP-40 0.02% during 30 minutes at room temperature. Then the tissue is washed twice during 5 minutes with wash solution (PBS with MgCl2 2 mM, EDTA 5 mM, NP-40 0.02% and Na deoxycholate 0.01%) at room temperature. Staining developed during 12 hours with stain solution (PBS with MgCl2 2 mM, NP-40 0.02%,Na deoxycholate 0.01%, K3Fe(CN)6 5 mM K4Fe(CN)6 5 mM and X-gal 1 mg/ml) at 37° C. The stained tissue is thereafter washed with PBS and embedded in paraffin.

### RESULTS

Experimental Eels

Eels at the start of the experiment (control, CPE and HPC groups: n=63) measured 84±5 cm and weighted 1235±229 g (K=0.21±0.02). All eels were silver with EIs of 11.0±1.2 (range 8.3-13.2) and active migrants (62 in stage IV, 1 in stage V). Eels from the different groups did not show differences in BL and BW.

Morphometry

The Eye Index significantly increased (>8%) after 3 weeks for eels of the CPE-group (P<0.001) as well as the eels of the HPC-group (P<0.05; FIG. 12). After 4 weeks, this difference was even doubled to 16% for eels of both groups. Eels from the rest-group showed no significant difference with eels from the control-group indicating that there was no time-effect. Also the other indicator of silvering PFLI showed a significant increase for eels of the CPE-group after 4 weeks (>2%; P<0.05) and similarly for eels of the HPC-group, already after 2 weeks (P<0.05; FIG. 12).

Blood Parameters

All experimental eels showed a decrease of Hct and Hb. Hct in eels from the HPC-group remained stable but then dropped in week 4 eels with 30% (P<0.05). Hct in eels from the CPE-group dropped immediately with >20% in weeks 1 and 2 (P<0.05), and then >45% in week 3 and 4 (resp. P=0.05 and 0.06). Hct also dropped in resting eels in a comparable fashion as HPC-eels, with 30%. Similar changes occurred for Hb.

Internal Parameters

The GSI increased in the CPE group already after 1 week (46%, P<0.001) and rose more with every week (week 2: 76%, week 3: 136%, week 4: 199%). In the HPC-group, we found an eel with a GSI>2 after 1 week and one after 2 weeks. Since eels with such GSIs were not found in the control and rest-group, and not in silver eels from Lake Grevelingen in general, an increase in gonad mass is indicated. Indeed, the GSI in the HPC-group was found significantly higher after 2 weeks, but no differences occurred after 3 and 4 weeks.

Discussion

We found that Zebrafish ZF4 cells (fibroblast cell line) were not rejected by silver eels over a period of 4 weeks. We further found that Zebrafish ZF4 cells proliferate in the eels after transplantation (FIG. 13). Silver eels treated with these hormone producing cells showed morphological changes of the eye size and pectoral fins. These parameters increased demonstrating a rise in the level of silvering. Changes were similar for CPE as well as for HPC treated eels. Silver eels treated with hormone producing cells showed evidence of induced maturation.

### Example 3

### Fish Bladder as a Recipient for Protein Producing Cells

In the present invention it was established that fish bladders are a suitable environment for the attachment and growth of implanted cells. We performed in vitro and in vivo experiments. For in vitro experiments tilapias and catfish bladders were provided with ZF4 cells for the attachment. The ZF4 cells were transfected with a construct containing a gene for β-galactosidase. These cells were proven to attach rapidly to a fish bladder wall and remain alive and able to invade the bladder wall under culture conditions. To test whether the natural environment of the fish bladder is suitable for attachment and growth of cells we developed a protocol for implantation in the bladder of two species of fish which doesn't affect the health of the animals. We observed that the ZF4 cell suspensions were able to attach and infiltrate the bladder wall for up to 5 days under conditions where urine is produced. The experiments show that species foreign cells attach to the wall of the urinary bladder and remain alive for up to 5 days. We also tested implantation of human primary tumor cell lines in the fish.

GFP-β-galactosidase Stable Cell Line

The pMP2838 plasmid was used to make a ZF4 stable cell line that expresses a fusion protein between the green fluorescent protein (GFP) and the β-galactosidase protein. This plasmid was described by Bakkers (2000). Briefly: the gfpN-LacZ gene of the pUAS-gfpN_LacZ plasmid was taken out and used as replacement for the gfp gene in the pEGFP-C3 plasmid. So, a green fluorescent fusion protein with β-galactosidase activity (gfpN-LacZ) was expressed under control of the CMV promoter. The pMP2838 plasmid also contains the neomycin resistant (Neor) gene that allows the selection of positive clones with gentamicin (G418) as described above for making stable cell lines. In the same way ZF4 stable cell lines were generated with the same protocol as described above (transfected with Fugene 6 and selected with G418).

At the day of the injection cells were harvested and quantified to a total of 108 cells for each cell line. The 4 cell lines were mixed to equal cell concentrations and diluted in DMEM-F12 medium without serum to a final concentration of 20 million cells in one ml.

β-galactosidase Tissue Staining

The tissue is briefly rinsed in PBS (phosphate buffered saline) and fixed immediately with paraformaldehyde 1%-glutaraldeyde 0.1% in PBS with MgCl2 2 mM, EDTA 5mM and NP-40 0.02% during 30 minutes at room temperature. Then it is washed twice during 5 minutes with wash solution (PBS with MgCl2 2 mM, EDTA 5 mM, NP-40 0.02% and Na-deoxycholate 0.01%) at room temperature. Thereafter the tissue is stained during 12 hours with stain solution (PBS with MgCl2 2 mM, NP-40 0.02%, Na-deoxycholate 0.01%, K3Fe(CN)6 5 mM, K4Fe(CN)6 5 mM, and X-gal 1 mg/ml) at 37° C. The stained tissue is then washed with PBS and embedded in paraffin.

Preparing a Cell Suspension of ZF4 Cells

The flask with cultured cells was observed under the inverted microscope to check for contamination, cell density etc. The cells were harvested when the cell density is around 80% confluence. The medium was removed and the monolayer was washed 1 time with 3 ml of PBS. The PBS was moved throughout the flask to remove as much as possible all extra cellular proteins. The PBS is completely removed and 0.5 ml of 0.25% trypsin solution was added. The flask is incubated at room temperature for a few minutes and checked under the inverted microscope to make sure that the cells of the monolayer are loose from the flask. When a single cell suspension has been obtained, 5 ml of complete medium were added. The cell suspension is centrifuged during 8 minutes at 1200 rpm and washed twice with serum-free medium. The density of the cell suspension was determined by counting an aliquot with a haemocytometer. The cells were finally resuspend at a final density of 3.5×10⁶ cells/ml in DMEM/F12 medium with out 10% fetal calf serum or antibiotics.

In vitro Experiment

Tilapias and catfish were killed after heavy anaesthesia with 300 ppm MS222. Bladders were dissected and washed briefly with PBS. The bladders were cut in two parts to expose the internal part of the bladder, and cultured 12 hours at 28° C. in Dulbecco's modified Eagle medium (DMEM/F12) medium (GIBCO) with 10% Fetal calf serum, Penicillin 100 U/ml, Streptomycin 100 mg/ml and G4180.5 mg/ml. The next day 100 ml of the cell suspension were added to the bladder and left to settle for at least 30 minutes. The bladder with the cells were cultured at 28° C. in DMEM/F12 medium with 10% Fetal calf serum, Penicillin 100 U/ml, Streptomycin 100 mg/ml and G418 0.5 mg/ml during 20 to 30 days. The medium was changed every week and pictures were taken under the stereoscopic microscope with fluorescence, in order to detect the fluorescent cells. After the 20 or 30 days of culture the tissue was fixed and stained for b-galactosidase activity as described above. Some representative results are shown in FIG. 15.

In vivo Experiment

The cell suspensions were prepared at different concentrations:
Pancreas Ewins cells at a concentration of 1×10⁷ and 1×10⁶
EGFP-β-Galactosidase ZF4 stable cells at concentration of 5×10⁵ and 1×10⁵
Mixture of EGFP-βGalactosidase, LHβ, FSHβ, a and FSHα+β ZF4 stable cells cells at concentration of 5×105 and 1×105

Tilapias were anesthetized with 300 ppm MS222 and the bladder was canulated with the use of a catater (PE diameter 0.8 mm). A description of the method used for inserting the canule is shown in FIG. 14. We washed with PBS and one ml of cell suspension was introduced into the lumen. The tilapias were allowed to rest for 30 minutes on their backs and were thereafter put back in the water where they recovered quickly. To test whether this technique also works with other fish species we performed the experiment shown in FIG. 14 on African catfish (Clarias gariepines). The results show that this fish is also suitable for this implanatation technique. Also in this fish species the removal of the tip of the papilla is required for the method.

After 2 and 4 days of the cell transplantations urine samples were obtained by canulation of the bladder. For several days samples were collected. Urine sample volumes varied from 100 microliter to >5 milliliter. The urine samples were frozen for further experiments. The fourth day after the implantation of cells the fish were sacrificed by 300 ppm MS222 followed by cervical dislocation. Bladder samples were further analysed for cell adhesion and metabolic activity as shown in FIG. 16.

Discussion

The results show that our technology for implantation of cells is very successful and can be performed standardly. Cells effienctly attach themselves to the bladder of the host fish. Furthermore, cells survive the presumably rather harsh environment of the fish urine and are able to grown and remain metabolically active as shown by the production of b-galactosidase.

On basis of these successful experiments we propose a suitable method for optimizing collection of urine samples as follows:

After cell implantation and some days of cell growth (e.g. 4 days as shown in our protocol) fish are maintained for several days in water containing 1% sodium chloride (300 mosm, the time depending on the fish species, for tilapia at least 3 days is possible without harming the health of the fish). This will result in very low urine production. After transferring the fish to fresh water (i.e. without salt) a high urine production follows that can subsequently be collected by a canule (this can be attached permanently to the fish as mentioned in the legend of FIG. 14). In this way a controlled product release is achieved. Since urine has a very low content of contaminating proteins and represents a normally sterile environment, there is little efford involved in purifying a peptide product produced by the implanted cells. For instance, a simple dialysis procedure allows to get rid of the contaminating (low molecular weight) urine waste products. A simple concentration using a centrifuge tube containing a molecular weight filter (e.g. Centricon technology) already delivers a rather pure product. Therefore this method can be considered extremely cost effective compared to purification of products produced by cell cultures. These cells cultures contain growth factors that are hard to get rid of. Another advantage of our method is that we don't need to add artificial growth factors. Such growth factor, such as fetal calf serum can be contaminated with diseases such as prion disease that are highly unwanted for any application and very difficult to get rid of. In contrast fish products are generally considered save for consumption of medical applications (e.g. there are no prion related disease known in fish that can be transferred to other organisms). Therefore for any consumption of medical applications our method can be of great advantage as well in costs as in quality over in-vitro cell cultures.

We also tested human cell implantations. This makes it possible to produce peptides that are post-translationally modified in a human fashion and therefore optimally suited for human biomedical applications. Although we only tested a human primary pancreas tumor cell line, it could be more advantageous to use human bladder tumor cells that are better adapted to the bladder environment. The long term survival of human cells after implantations in fish has been recently shown by Lee et al, (Dev. Dynamics, 2333:1560-1570, (2005) who showed that human melanoma cells can proliferate in zebrafish for at least 3 months after implantation. These findings also underscore the possibility to use fish as a screening system for anticancer drugs. With our invention, we add the possibility that the presence of the tumor cells can be detected in the fish by a simple analysis of the urine for the presence of human peptides. Since human peptides can easily be discriminated from fish peptides by standard mass spectrometric analysis, we propose the following method:

After implantation with human tumor cells in the bladder, using the method as shown in FIG. 14, urine analysis indicates whether the tumor cells are still present. After the addition of potential antitumor drugs to the water of the fish (many compounds are taken up by fish through the gils or mouth), the urine tests show whether the drug are effective. Alternatively, if the chemical compound is expected to be poorly transmissible through tissue, then add the compound into the bladder via the canule (as described in FIG. 14). Samples of the urine after treatment with the antitumor drug will be analyzed for the presence of human peptides providing a simple read-out method for the activity of the drug on the tumor cells. The advantage of this method over screening of antitumor compounds on cell cultures, is that in our invention the cells are in a natural tissue environment in the absence of artificial growth factors (such as fetal calf serum) and therefore the effect of the antitumor drug is more predictive for the effect on tumors in patients.

In a preferred embodiment the ZF4 cells produce a fluorescent protein that makes it possible to analyse the presence of the cells in a transparent organisms such as fish embryos, transparent fish and frogs, or any other transparent animal.

### References

Adachi, S., Ijiri, S., Kazeto, Y., Yamauchi, K., 2003. Oogenesis in the Japanese Eel, Anguilla japonica. In: Aida, K., Tsukamoto, K., Yamauchi, K., (Eds.), Eel Biology, Springer, pp. 502-518.
Durif, C., Dufour, S., Elie, P. (2005). The silvering process of the eel: a new classification from the yellow resident stage to the silver migrating stage. Journal of Fish Biology 66: 1-19.
Jonge, H. W. de, Bakker, M. A. G. de, Verbeek, F. J., Weijs, W. (2005).
Embedding of large specimens in glycol methacrylate: prerequisites for multi-signal detection and high-resolution imaging. Microscopy research and technique 66: 25-30.
Palstra A P, Cohen E G H, Niemantsverdriet P R W, van Ginneken V. J. T., van den Thillart, G. E. E. J. M. (2005) Artificial maturation and reproduction of European silver eel: Development of oocytes during final maturation. Aquaculture 249 (1-4): 533-547.
Pankhurst, N. W. (1982). Relation of visual changes to the onset of sexual maturation in the European eel Anguilla anguilla (L.). J Fish Biol. 21: 127-140.
Verslycke, T., Vandenbergh, G. F., Versonnen, B., Arijs, K., Janssen, C. R. (2002). Induction of vitellogenesis in 17α-ethinylestradiol-exposed rainbow trout (Oncorhynchus mykiss): a method comparison. Comparitive Biochemistry and Physiology Part C 132: 483-492.
Versonnen, B. J, Goemans, G., Belpaire, C., Janssen, C. R. (2004). Vitellogenin content in European eel (Anguilla anguilla) in Flanders, Belgium. Environ. Pollut. 128(3): 363-371.

### BRIEF DESCRIPTION OF THE DRAWINGS

FIG. 1. Dendrogram based on alignment of the LHβ (LHβ), FSHβ (FSHβ) originating from a (a) of human (Hs), mice (Mm), rat (Rn), zebrafish (Dr) and eel (Aj).

FIG. 2. p3XFLAG-CMV-9 expression vector

FIG. 3. Cloning strategy for the expression of LHβ

FIG. 4. The predicted aminoacid sequence of LHb gives a protein of 187 aminoacids with a molecular weight of 20593.8

FIG. 5. Cloning strategy for the expression of FSHβ

FIG. 6. The predicted aminoacid sequence of FSHbblu and FSHb gives a protein of 174/183 aminoacids with a molecular weight of 19062.24/19965.21 respectively to the two different strategies of cloning.

FIG. 7. Cloning strategy for the expression of α.

FIG. 8. The predicted amino acid sequence gives a protein of 190 amino acids with a molecular weight of 21083.24.

FIG. 9. Transfection of ZF4 cells with PEYFP-Ni. Transfection is observed after 24 h of transfection.

FIG. 10. Immunohistochemistry with anti-FLAG antibody in cotransfected cells with pEYFP-N1.

FIG. 11. Western blot with anti-FLAG. The antibody recognized a protein of the expected size for FLAG-BAP.

FIG. 12 Changes (%) in Eye index (El; panel A) and Pectoral Fin Length Index (PFLI; panel B) with regard to the initial stage of silver eels treated with either CPE or HPC during 4 weeks (W1-W4). The Eye index (EI) increases equally for both groups, a similar change can be observed for the Pectoral Fin Length Index (PFLI).

FIG. 13 Suspensions with β-galactosidase expressing ZF4 cells were injected subcutaneaously in silver eels. Tissue samples were stained for β-galactosidase (blue) using X-gal and showed a progress of cell intensity in the injected area.

FIG. 14. In-vivo experiment in tilapia bladder.

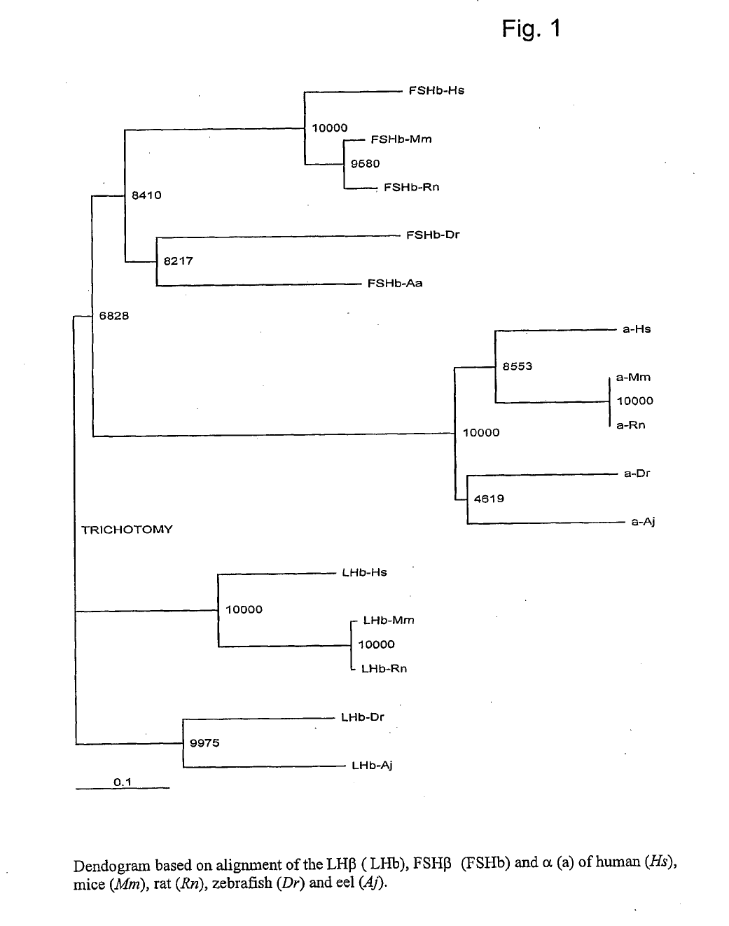

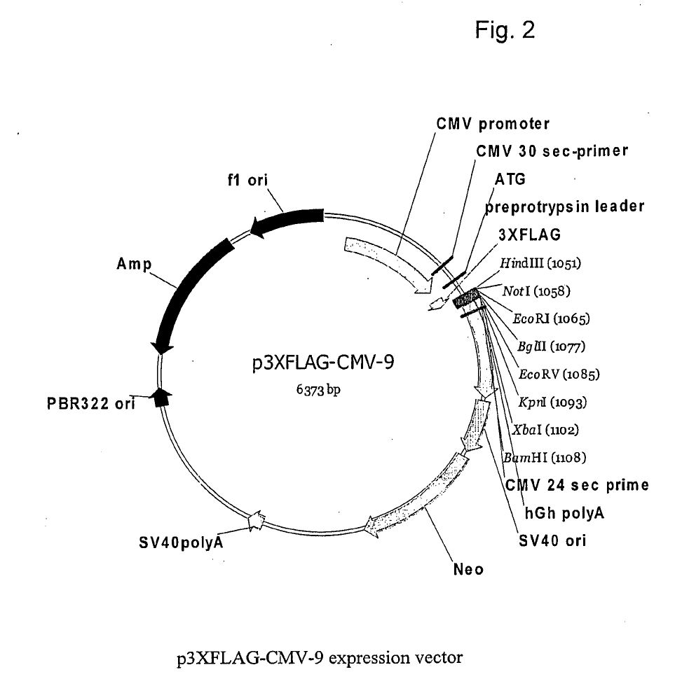

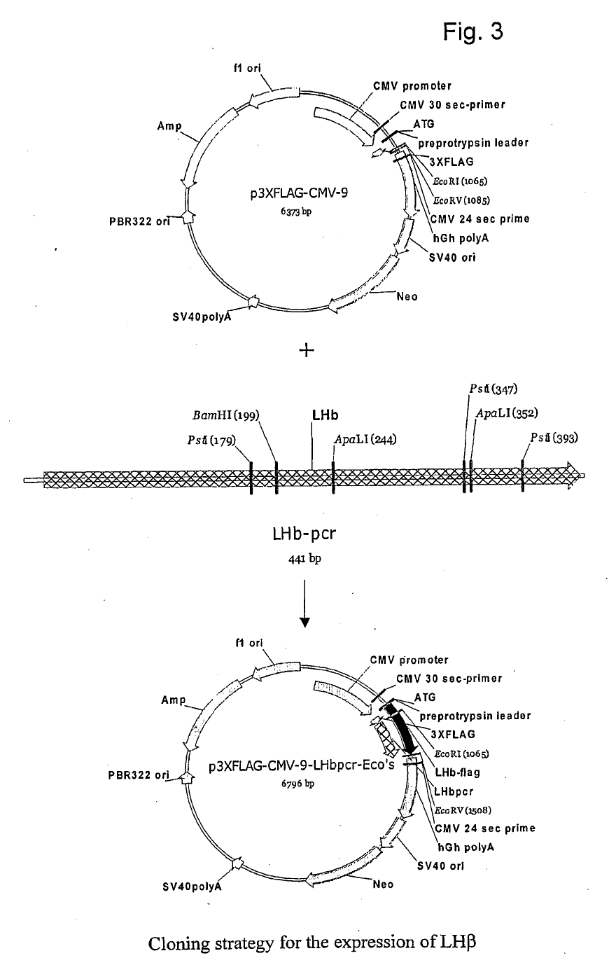

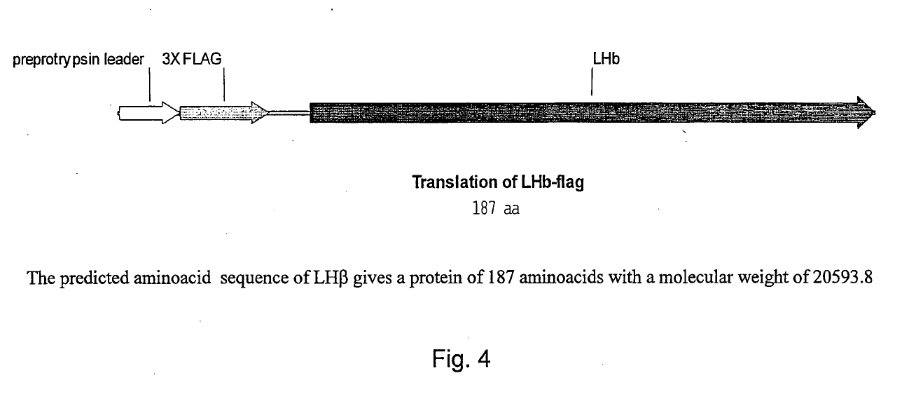

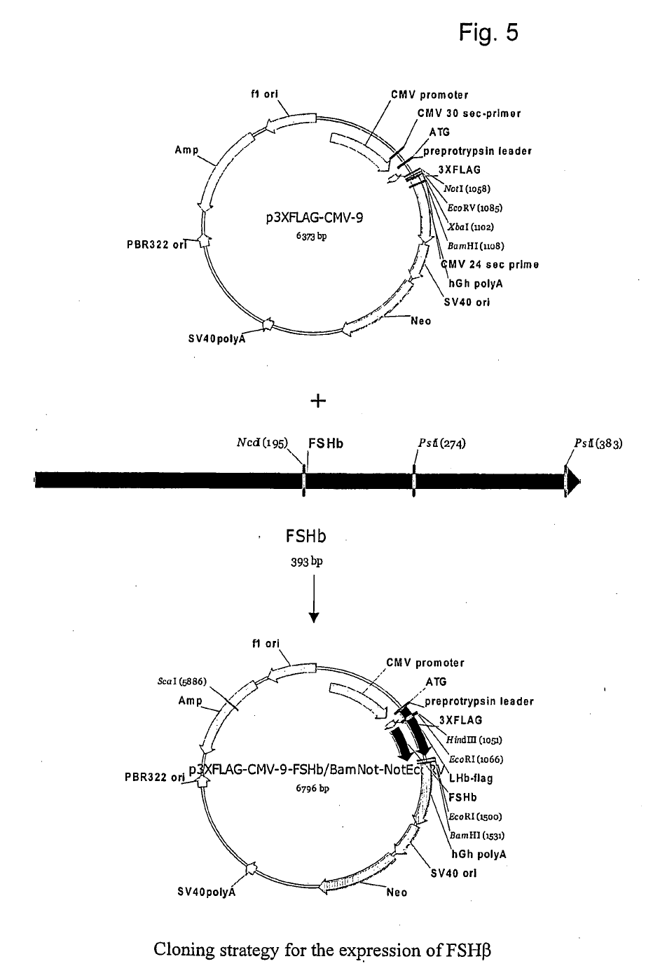

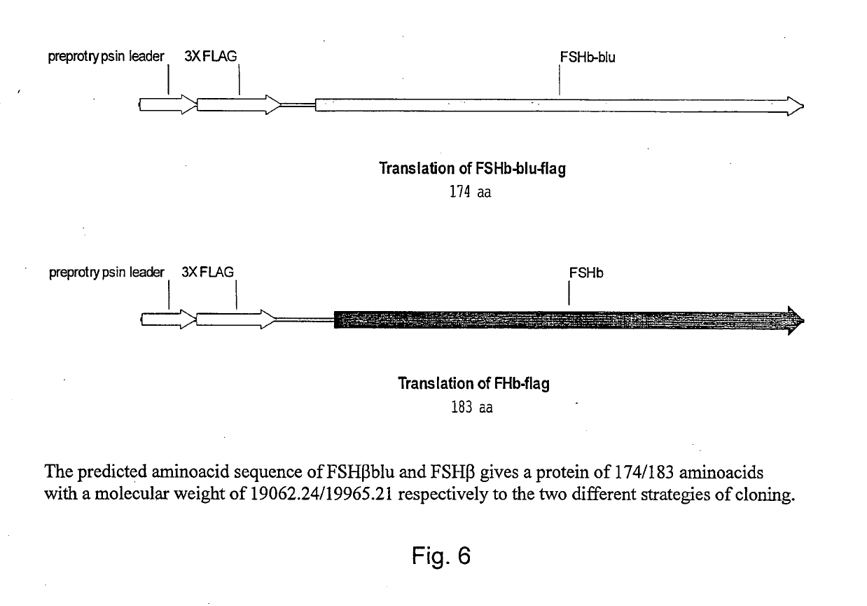

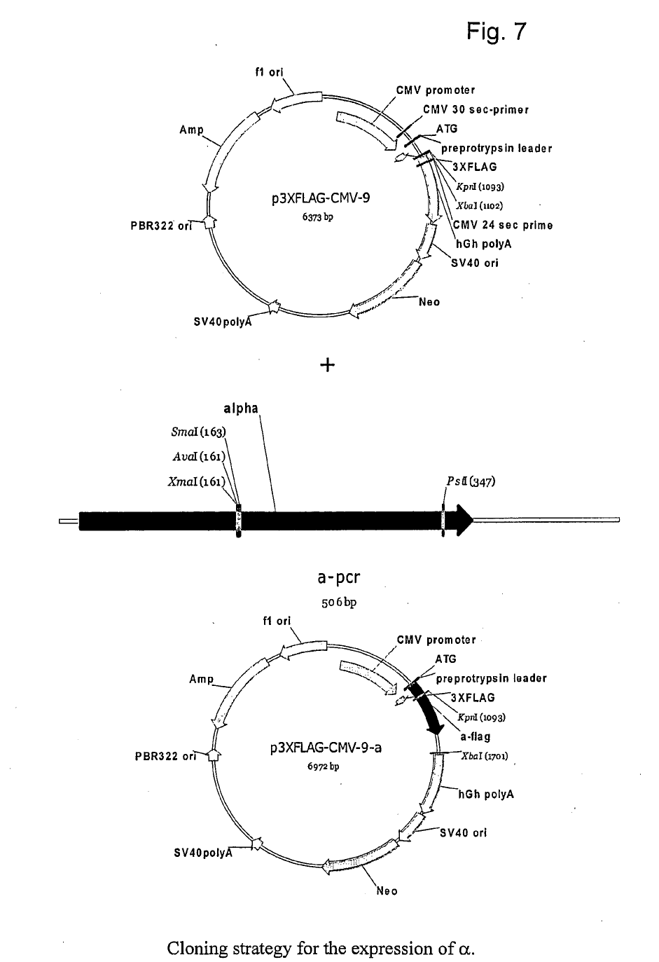

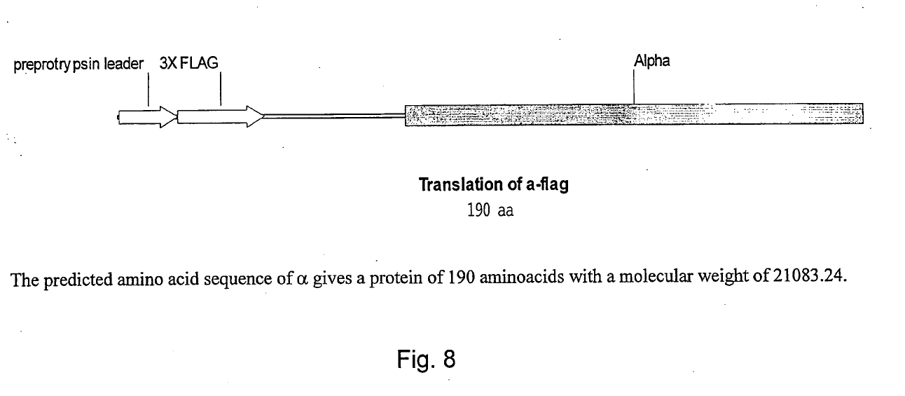

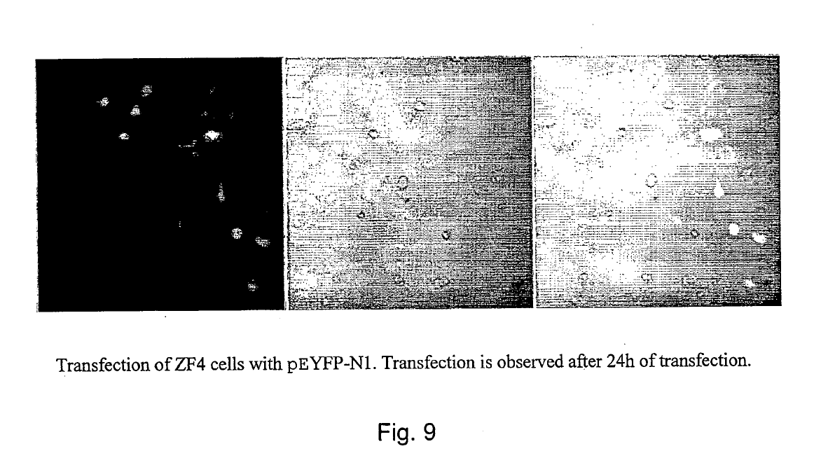

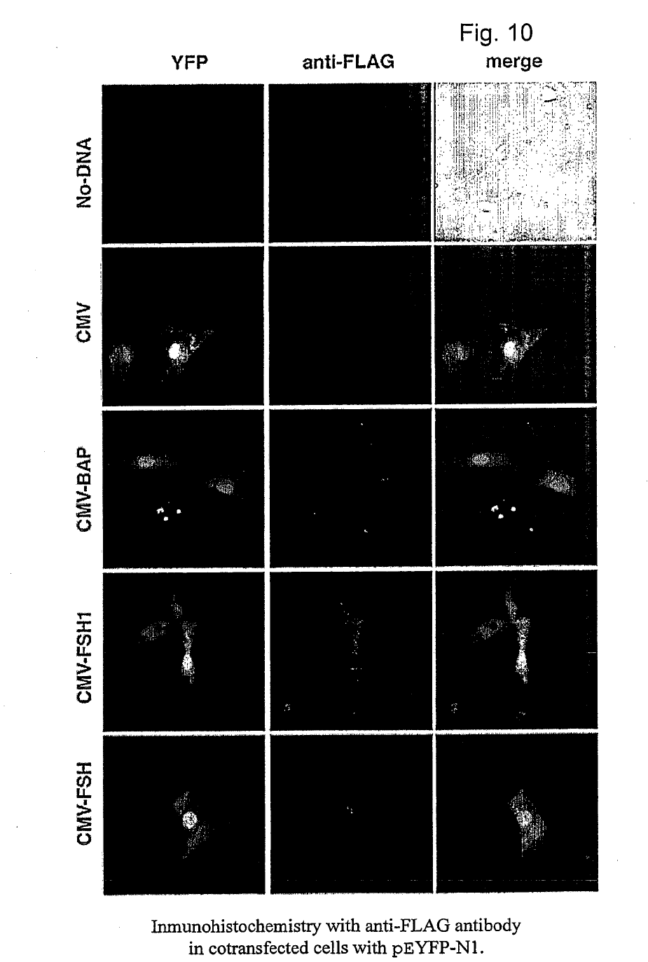

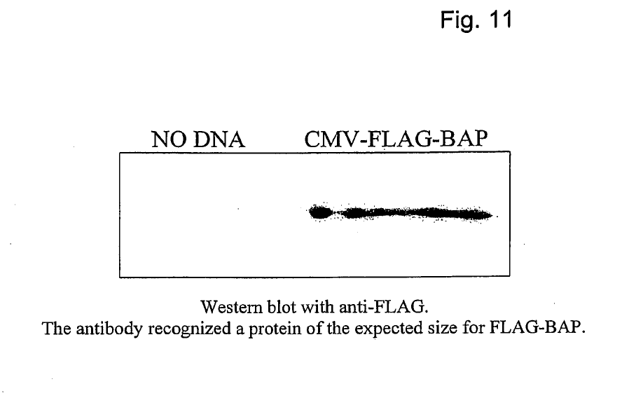

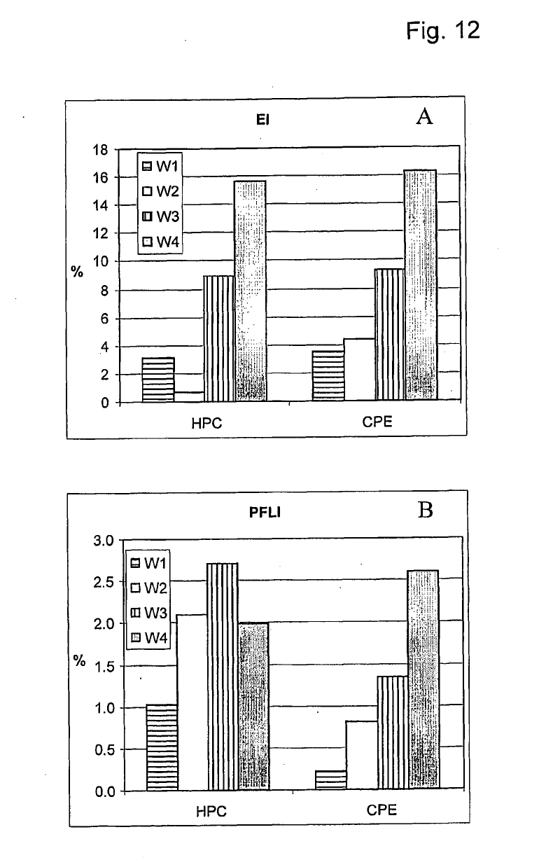

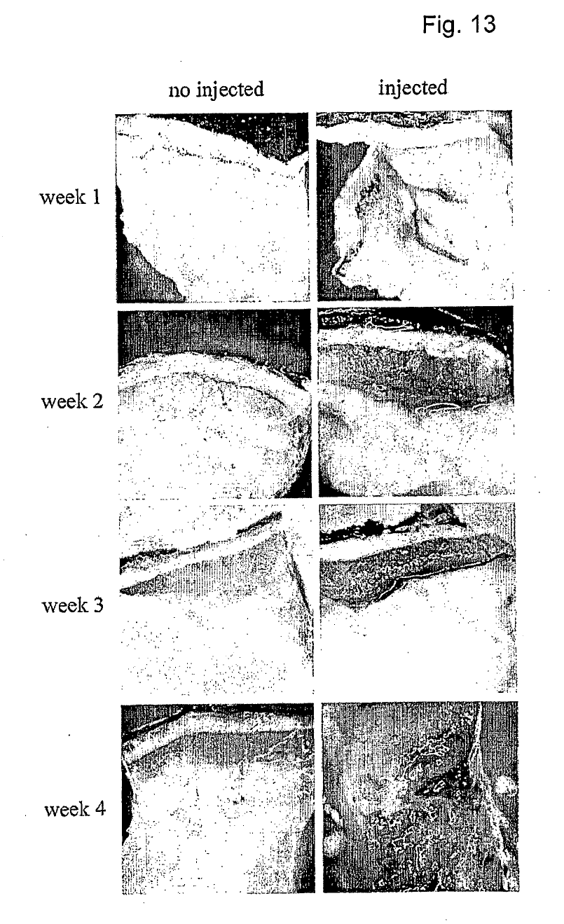

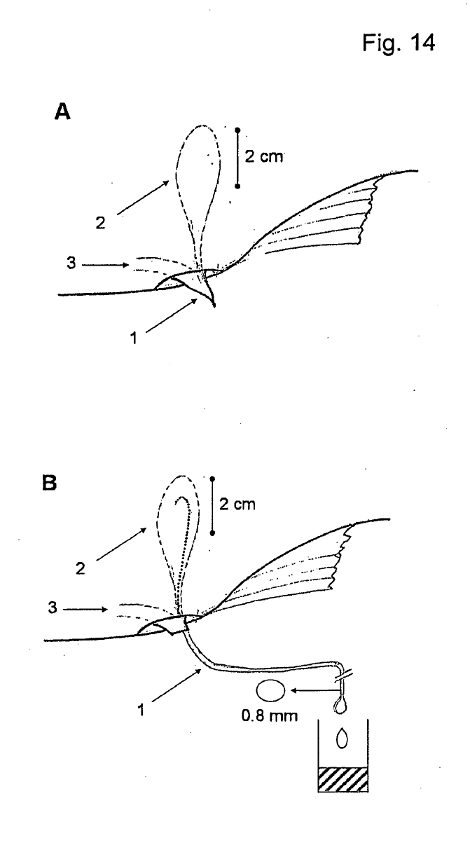

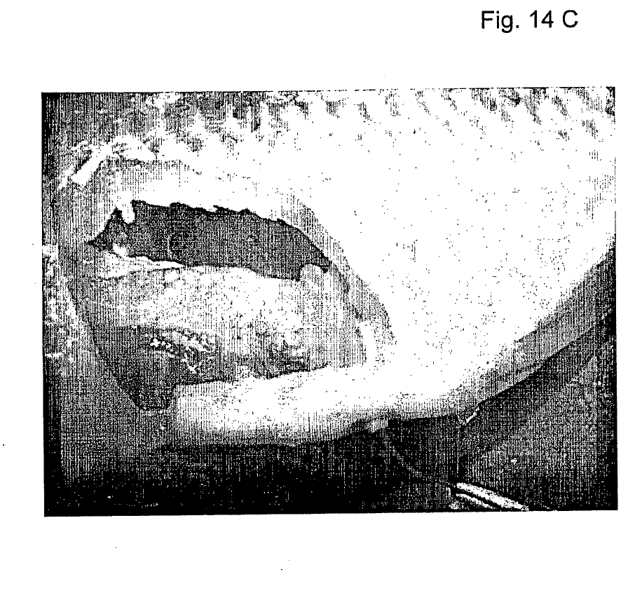

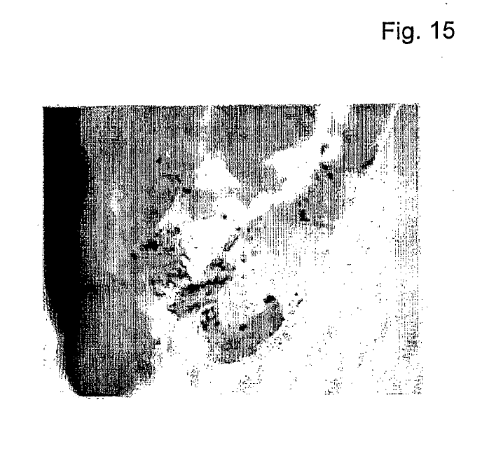

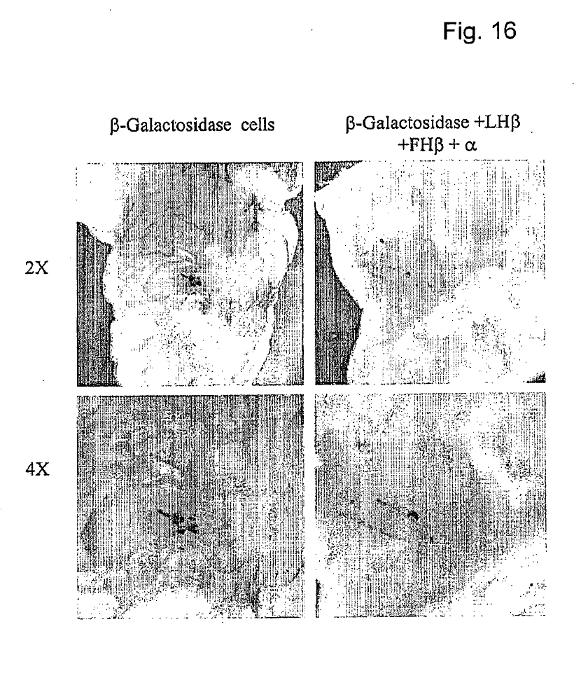

A. A schematic presentation of the location of the bladder (2) in the most caudal part of the abdomen. The intestine (3), and the urethra exit together at the papilla (1). The arrow indicates the place where the tip has to be cut in order to get acces to the urethra, which leads into the urinary bladder.

B. A thin PE tube (1) can be fed trough the urethra into the bladder (dashed line). The tip of the canula needs to be rounded to prevent wall damage at the intrusion, and needs small side holes for urine drainage.

C. The picture shows the location of the cannula in the urinary bladder in a freshly killed tilapia. With little effort the canula can be fixed (with sutures) to the rays of the anal fin, where it can stay for weeks. A permanent canula allows bladder drainage at any time. A preferable setting is when the fish is placed in a flow-box, and the canula is attached to a collecting vessel on one side of the box

FIG. 15 In-vitro experiment in tilapia bladder. Urinary bladder of tilapia was incubated with β-gal ZF4 cells. The cells attached to the wall within 30 minutes, and invaded the host tissue. The protein producing cells attached to the bladder wall stand out by the blue color.

FIG. 16. In-vivo experiment in tilapia bladder. Light microscopy pictures from urinary bladder of tilapias injected in the lumen with ZF4 cells transfected with gene constructs for β-galactosidase. The bladders were dissected 5 days after the injection and show that a large part of the bladder contains the foreign cells (colored blue).

1. A method for obtaining a peptide produced by cells, said method comprising
providing a non-human animal with at least one of said cells, and
harvesting said peptide from said non-human animal.

2. (canceled)

3. The method according to claim 1, wherein said peptide is selected from the group consisting of Luteinizing hormone (LH), Follicle stimulating hormone (FSH), Chorionic gonadotropin (CG), Growth hormone, Corticoliberin (Adreno corticotropic hormone), Thyroid stimulating hormone, Follicle-stimulating hormone (FSH), Luteinizing hormone (LH), Prolactin, Chorion gonadotropin (CG), MG (Menopause gonadotropin), Somatotropin, Prolactin, Placental lactogen, Calcitonin, Parathyroid hormone, Colony-Stimulating Factor (CSF), Epidermal growth factor (EGF), erythropoietin, and any other peptide that is either a hormone, enzyme, antibody, or inhibitor from any animal.

4. The method according to claim 1, wherein said peptide is human.

5. The method according to claim 1, wherein said cells are genetically modified to express said peptide.

6. The method according to claim 1, wherein said peptide is of the same genus as said non-human animal.

7. The method according to claim 5, wherein said cells have been provided with one or more genes encoding said peptide.

8. The method according to claim 1, wherein said cells are a clonal population.

9. The method according to claim 1, wherein said cells have been selected for a reduced immunogenicity in said non-human animal.

10. The method according to claim 1, wherein said non-human animal is a fish, an amphibian or a mammal.

11. The method according to claim 10, further comprising:
keeping said non-human animal at a temperature at which said non-human animal's immune system is dysfunctional.

12. The method according to claim 1, wherein said cell is provided to the bladder and/or gut of said non-human animal.

13. (canceled)

14. The method according to claim 1, wherein said cell is a tumor cell.

15. The method according to claim 1, wherein said cell is a mammalian cell.

16. A cell that is suited for transplantation into a non-human animal's bladder or gut, wherein said cell is a mammalian, fish, or amphibian cell genetically modified to produce a peptide.

17. (canceled)

18. (canceled)

19. The cell of claim 16, in situ, within a bladder or gut of a non-human animal.

20. The cell of claim 19, wherein the non-human animal is a fish or an amphibian.

21. The cell of claim 20, wherein the non-human animal is transparent.

22. The cell of claim 19, wherein the non-human animal is selected from the group consisting of a carp, salmon, goldfish, zebra fish, trout, catfish, sturgeon, sea bream, sea bass, tilapia, medaka, Nile perch, glass catfish and a commonly cultured aquaria fish.

23. (canceled)

24. A fish and/or amphibian comprising a cell implant in the bladder or gut of said fish and/or amphibian.

25. The fish and/or amphibian according to claim 24, wherein said cell implant comprises a mammalian cell.

26. The fish and/or amphibian according to claim 24, wherein said cell implant comprises a tumor cell.

27. A method for transplanting cells into a fish, said method comprising
anaesthetizing said fish,
opening the papilla connecting the fish's bladder and/or intestines with surrounding water, and
injecting cells via the urethra and/or intestine into the intestinal and/or bladder lumen.

28. The method according to claim 27, wherein said papilla is cut to remove the tip of papilla.

29. A fish having a papilla connecting the fish's bladder and/or intestines with surrounding water, wherein the tip of said papilla has been removed.

30. The fish according to claim 29, comprising a canule.


SEQUENCE LISTING (17 sequences)

SEQ ID NO: 1 - DNA, 958 bp, Danio rerio
  CDS (67)..(489): LH beta
atatataaatctggacacgcagagacacttacaacagcctgctgagcaaccgcaacgcctgtcaagatgttattggctggaaatggtgtcttctttctcttctctttgtttttcctgctggcggctgctcagagcttggtttttccacgctgtgagctagtaaatgagacggtatcggtggaaaaagagggctgtccaaaatgcctggtgtttcagaccaccatctgcagcggccactgcgtaacaagggatcccgtttacaagagcccgttttccaccgtccaccagacagtgtgcatgtaccgggacgtccgctatgagaccattaacctgcccgactgttccgccggcgtggacccgcagatcacatacccggtggcgctgagctgcgactgcagtctgtgcaccataaacacttccgactgcaccatccagagcctgcagcccgacttctgcatgtcccagagagaggatttccccgcatactagacctcgggcaactcacgtcaacctacgcacatagtcgagctcagcattattagccctcctgtatgttttttccattaatatatatactttcaagacactagtattcagcttaaagtgacatttaaagactaaactaggttaattagggggaaaagtagagtaagtcattgtataatagtggtttgttctggagacaatccaaaactaatattgcttaagggggctaataaaattgaccttaaaatgaatttaaataatttaaaaactgcatttattctagtcgaaataaaagaaataagactttctttagaagaaaaaacattataggaaatactgcaaaaaaattcctgaatctgttcaacatcattcgggaaatcaaaggagggctaataactgtgacttcagctgtacatcaataaagaggctggttcttaaattcaaaaaaaaaaaaaaaaaaaaaaaaaaaaaa

SEQ ID NO: 2 - protein, 140 aa, Danio rerio
MLLAGNGVFFLFSLFFLLAAAQSLVFPRCELVNETVSVEKEGCPKCLVFQTTICSGHCVTRDPVYKSPFSTVHQTVCMYRDVRYETINLPDCSAGVDPQITYPVALSCDCSLCTINTSDCTIQSLQPDFCMSQREDFPAY

SEQ ID NO: 3 - DNA, 1038 bp, Danio rerio
ggtctccacgaaactcccgcagatgaggatgcgtgtgcttgttctggcgctgctgttgccggtgttaatgagcgcagaatcagaatgcaggtgcagctgtcgactcaccaacatctccatcactgtggagagcgaagaatgtgggagctgcgtcacaatcgacaccacagcctgtgcaggactatgctggacaatggatcgagtttaccctagttccatggcacagcacacccagaaggtctgtaacttcaagaacttgatgtacaagagctacgagtttaaaggctgtcctgcaggggttgattcagtcttcgtgtaccccgtggctctgagctgtgagtgcaaccaggttaactcagacacaacagactggggagctatcagcccgcagaccaccagctgcagcatacactagagcactgtatcatgaccttaacaacatgtacgttgcagaatcaaattaagtaaggagtacaattagaccatttaaggatatcaattatttacaaaacctttagtttttcatgcatcccacacacatggtaatttggttacttgaattaatctgttgtgttaattctatagttggtactatggtaactagagtactagagtatccaatgctatactagttttaattacagttaattatagaaaagtatgctacagtatttattacagtttttctgttttcaatatttagtactacagtatgctagtgcattcattaacaataagctgtaaatactataataaatacaggttaatacactttactatagtatgcttgatcaacactattatttaatgtgagttactatagtacttttcaattgggatttgtcattttggatattgtgggcttttttggctattcataaagttttttttattttttttttatttaattttcagtcaaatggaaacaagtccaccataatacacttgtgtttcttttgtcaaacttatcaatttgtgtctgtagatttcaattacaatacatattttaaaggccaaaaaaaaaaaaaaaaaaaaaaa

SEQ ID NO: 4 - DNA, 710 bp, Danio rerio
  CDS (46)..(399): FSH/LH alpha
gaagacactcatcacgctccgccggaagtcgaggacaaagccatcatgttttggacaagatacgctgaagcaagcattttcttgttgttaatgattcttcatgtcggacaactgtattcaagaaacgatgtgtctaactatggatgtgaagagtgcaaactcaagatgaacgaacgtttctccaaacccggggctccggtctatcagtgcgtgggctgctgcttttcgagagcttaccccacacccctgaggtccaagaaaaccatgcttgtcccaaaaaacatcacatcagaagccacttgctgtgtagcaaaagaatctaaaatggttgccacgaatatcccactatacaaccacacagactgccactgcagcacctgttactatcataagtcttaaaacacactctcttcacatttctcaaatgctcatttcctgttcttaaatcacagtgactcatgaaatatgatttttatgtagctttccatatttcaactgtggccatttccaattcgtttctaaaatggttggcataagtattgtaaactgcatattctgtcactatccctttaagagcgtaatatgccatcctttactatcattaaatcgcttatttattttgttgcctttactgtgacattcttcaaatctataaatgaaataaaagattgctgaaggcaaaaaaaaaaaaaaaaaaaaaaaaaaaaaaa

SEQ ID NO: 5 - protein, 117 aa, Danio rerio
MFWTRYAEASIFLLLMILHVGQLYSRNDVSNYGCEECKLKMNERFSKPGAPVYQCVGCCFSRAYPTPLRSKKTMLVPKNITSEATCCVAKESKMVATNIPLYNHTDCHCSTCYYHKS

SEQ ID NO: 6 - DNA, 27 bp, Artificial Sequence
  Primer Upper LH-beta/EcoRI
caaccgaattcaacgccttcaagatgt

SEQ ID NO: 7 - DNA, 24 bp, Artificial Sequence
  Primer Lower LH-beta/EcoRV
ccgatatctagtatgcggggaaat

SEQ ID NO: 8 - DNA, 20 bp, Artificial Sequence
  Primer Upper FSH-beta3
aggatgcgtgtgcttgttct

SEQ ID NO: 9 - DNA, 25 bp, Artificial Sequence
  Primer Lower FSH-beta 2/3
tgttgttaaggtcatgatacagtgc

SEQ ID NO: 10 - DNA, 20 bp, Artificial Sequence
  Primer Upper-alpha1
gtcgaggacaaagccatcat

SEQ ID NO: 11 - DNA, 21 bp, Artificial Sequence
  Primer Lower-alpha1
tgccaaccattttagaaacga

SEQ ID NO: 12 - DNA, 20 bp, Artificial Sequence
  Primer Upper-Activin A
tgctgcaagcgacaatttta

SEQ ID NO: 13 - DNA, 19 bp, Artificial Sequence
  Primer Lower-Activin A
cattcgtttcggactcaag

SEQ ID NO: 14 - DNA, 20 bp, Artificial Sequence
  Primer Upper-Activin B
caacttagatggacacgctg

SEQ ID NO: 15 - DNA, 20 bp, Artificial Sequence
  Primer Lower-Activin B
gtggatgtcgaggtcttgtc

SEQ ID NO: 16 - DNA, 20 bp, Artificial Sequence
  Primer Upper-beta Actin
ccccttgttcacaataacct

SEQ ID NO: 17 - DNA, 19 bp, Artificial Sequence
  Primer Lower-beta Actin
tctgtggctttgggattca